(19) United States
(12) Patent Application Publication
(10) Pub. No.: US 2006/0235532 A1
(43) Pub. Date: Oct. 19, 2006

(54) Unit for treatment of the degeneration of an intervertebral disc

(75) Inventors: Alain Meunier, Paris (FR); Jacques Senegas, Merignac (FR); Regis Le Couedic, Andresy (FR)

Correspondence Address: CABINET BEAU DE LOMENIE, 158 RUE DE L'UNIVERSITE, PARIS, 75007, FR

(73) Assignee: ABBOTT SPINE, Bordeaux (FR)

(21) Appl. No.: 10/542,607

(22) PCT Filed: Dec. 29, 2003
(86) PCT No.: PCT/FR03/03929

§ 371 Date: Mar. 17, 2006

(30) Foreign Application Priority Data
Jan. 20, 2003 (FR) 0300555

(51) Int. Cl. A61F 2/44; A61B 17/70

(52) U.S. Cl. 623/17.16; 606/61

(57) ABSTRACT

The invention relates to an assembly for treating degeneration of a damaged intervertebral disk disposed between two vertebrae. The treatment assembly includes cells that may be analogous to those of the intervertebral disk, and that are suitable for implanting in said disk; and an intervertebral implant. The intervertebral implant includes an intervertebral spacer for placing between said vertebrae, and a fastener device for fastening said spacer on said vertebrae.

### FIELD OF THE INVENTION

The present invention relates to an assembly for treating degeneration of an intervertebral disk.

### DESCRIPTION OF THE RELATED ART

As is well known, an intervertebral disk serves to unite two vertebral bodies and has the shape of a biconvex lens which fits against and attaches to the articular surfaces of the vertebral bodies. Such disks are constituted by two portions: a peripheral portion that is very hard and of very compact texture, and a central portion that is a soft and gelatinous substance.

For various reasons, intervertebral disk pathologies can lead to more or less marked degeneration of the disk which can then no longer perform its normal function between the vertebrae.

Faced with such a lesion or degeneration of an intervertebral disk, it is necessary to apply a surgical technique in an attempt to obtain regeneration of the disk and thus normal biomechanical behavior thereof.

One technique for attempting to obtain such regeneration consists in implanting cells in the damaged disk for the purpose of regenerating the disk, which cells may be of the same nature as those constituting the disk, or of some other nature.

Nevertheless, it can be expected that such a technique that does no more than introduce new cells into a biomechanical environment that is already degraded will not be very effective, with the new cells being subjected to excessive stress, thus compromising their own long-term survival. It can therefore be seen that such treatment has little chance in achieving the expected results, i.e. regeneration of the intervertebral disk.

It is therefore important to define means enabling effective regeneration of the intervertebral disk to be obtained in a manner that is long-lasting. That is the object of the present invention.

### SUMMARY OF THE INVENTION

The inventors have found that after regeneration cells have been implanted in a damaged intervertebral disk, the damage or necrosis of the cells constituting the disk does not disappear, or does not disappear sufficiently, because of the mechanical pressure that is exerted on the disk by the surrounding vertebrae. Furthermore, the inventors have also found that the origin of some of the degeneration of cells in the intervertebral disk can be of purely mechanical origin, or that this purely mechanical origin can lead to conditions that encourage degeneration of the disk for other biological damage.

As mentioned above, the object of the invention is achieved by an assembly for treating degeneration of a damaged intervertebral disk disposed between two vertebrae, which assembly is characterized in that it comprises cells optionally analogous to those of the intervertebral disk and suitable for implanting in said disk; and an intervertebral implant comprising an intervertebral spacer for placing between said vertebrae in order to limit the stresses applied to said disks, and fastener means for fastening said spacer to said vertebrae.

It will be understood firstly that the cells implanted in the intervertebral disk enable cells of the intervertebral disk to regenerate, and secondly that this regeneration is made possible because of the presence of the installed intervertebral implant having its spacer disposed between the processes of the vertebrae, thereby maintaining the spacing between them and thus limiting the mechanical stresses that are applied to the intervertebral disk during treatment.

The implantable cells that are used may be obtained by various methods, such as taking cells from an intervertebral disk of the patient, taking adult stem cells from the bone marrow of the patient, or indeed taking embryonic stem cells.

In addition, the treatment assembly advantageously includes means for injecting cells into the disk, said injection means possibly being of the canular-syringe type.

Also preferably, the spacer of the intervertebral implant comprises a central portion and two end portions, each end portion having a groove defined by two limbs, each of said limbs being suitable for receiving the spinus process of one of the vertebrae beside the disk to be treated.

Other characteristics and advantages of the invention appear better on reading the following description of embodiments of the invention given as non-limiting examples.

### BRIEF DESCRIPTION OF THE DRAWINGS

The description refers to the accompanying drawings, in which:

FIG. 1 shows an assembly for treating degeneration of an intervertebral disk in accordance with the invention;

FIG. 2 is a perspective view of an intervertebral implant usable in the present invention; and

FIG. 3 is a vertical section view of the implant shown in FIG. 2.

### DETAILED DESCRIPTION OF THE INVENTION

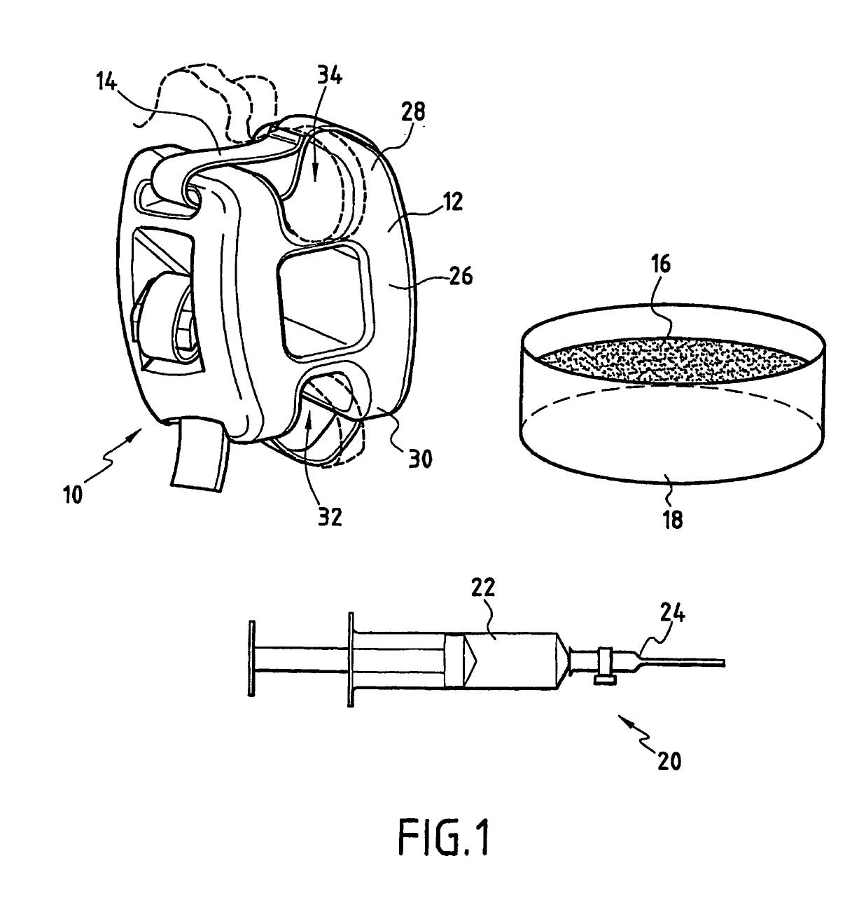

FIG. 1 is a simplified diagram showing an intervertebral implant 10 comprising a spacer 12 with fastener means 14; injectable cells for regenerating the intervertebral disk, the cells being symbolically referenced 16 and shown in a culture disk 18; and injector means 20 for injecting the cells 16 into the intervertebral disk. The injector means 20 are constituted in this particular embodiment of the invention by a syringe body 22 and a canula 24.

As explained above in accordance with the invention the intervertebral implant 10 constituting a portion of the treatment assembly is intended to enable a given spacing to be set between the vertebrae adjacent to the intervertebral disk for treatment, thereby limiting the mechanical stresses that are applied to said disk. Furthermore, the treatment assembly includes injectable cells for cell therapy, with the cells being possibly injected or implanted at the same time as the implant is put into place, or subsequently, as explained below.

Intervertebral implants are themselves well known for their function of spacing vertebrae apart and holding them together via their processes. Such intervertebral implants are described in particular in the Applicants' PCT patent applications, and in particular by the patent applications WO 02/051326 and PCT/FR02/00888.

In general, as described below and as shown in FIG. 1, these implants are constituted by a spacer 12 comprising a central portion 26 and two end portions 28 and 30. The end portions 28 and 30 define respective grooves 32 and 34 for receiving the spinus processes of the vertebrae that are to be held at a substantially constant distance apart from each other. In general, and preferably, the central portion of the spacer 12 presents some capacity for elastic deformation so as to allow the vertebrae to move relative to each other while still ensuring they are suitably spaced apart.

The implantable cells usable for regenerating the intervertebral disk can have a plurality of origins:

they may be cells taken from the patient, from the disk in question and cultured in a suitable medium (possibly in three dimensions) such as an alginate bead, a porous structure of polylacticacid/polyglycolicacid (PLA/PGA), collagen foam.

It is also possible to use autologous adult stem cells taken from the bone marrow, e.g. in the iliac crest, and then placed in a culture medium identical to those mentioned above.

Finally, it is possible to use embryonic stem cells treated in the same manner as that mentioned above.

These cells can be implanted in the intervertebral disk for treatment at the same time as the intervertebral implant is being put into place, or during a completely separate operation performed before or after putting the intervertebral implant into place. In all configurations, similar results are obtained.

When performing surgery during which the implantation of cells in the intervertebral disk and the installation of the intervertebral implant takes place simultaneously, the operation is performed by implementing the following steps:

the patient on whom the operation is to be performed in placed in a ventral prone position. It is desirable to have a position with physiological lumbar lordosis;

the surgeon proceeds to expose the spinus processes and to disinsert the supraspinal ligament;

the spinus processes are prepared in particular by scraping the vertebrae concerned; and

culture cells are then injected into the intervertebral disk.

Preparations for the step of injecting cells into the intervertebral disk depends on the type of culture medium that has been used. It can be necessary to separate the cells from the medium prior to implanting them in the disk.

Thereafter, the surgeon measures the gap between the spinus processes of the vertebrae in order to determine the size of intervertebral implant spacer to put into place. Finally, the implant is put into place using the operating techniques that correspond to the selected implant, and then the approach is closed.

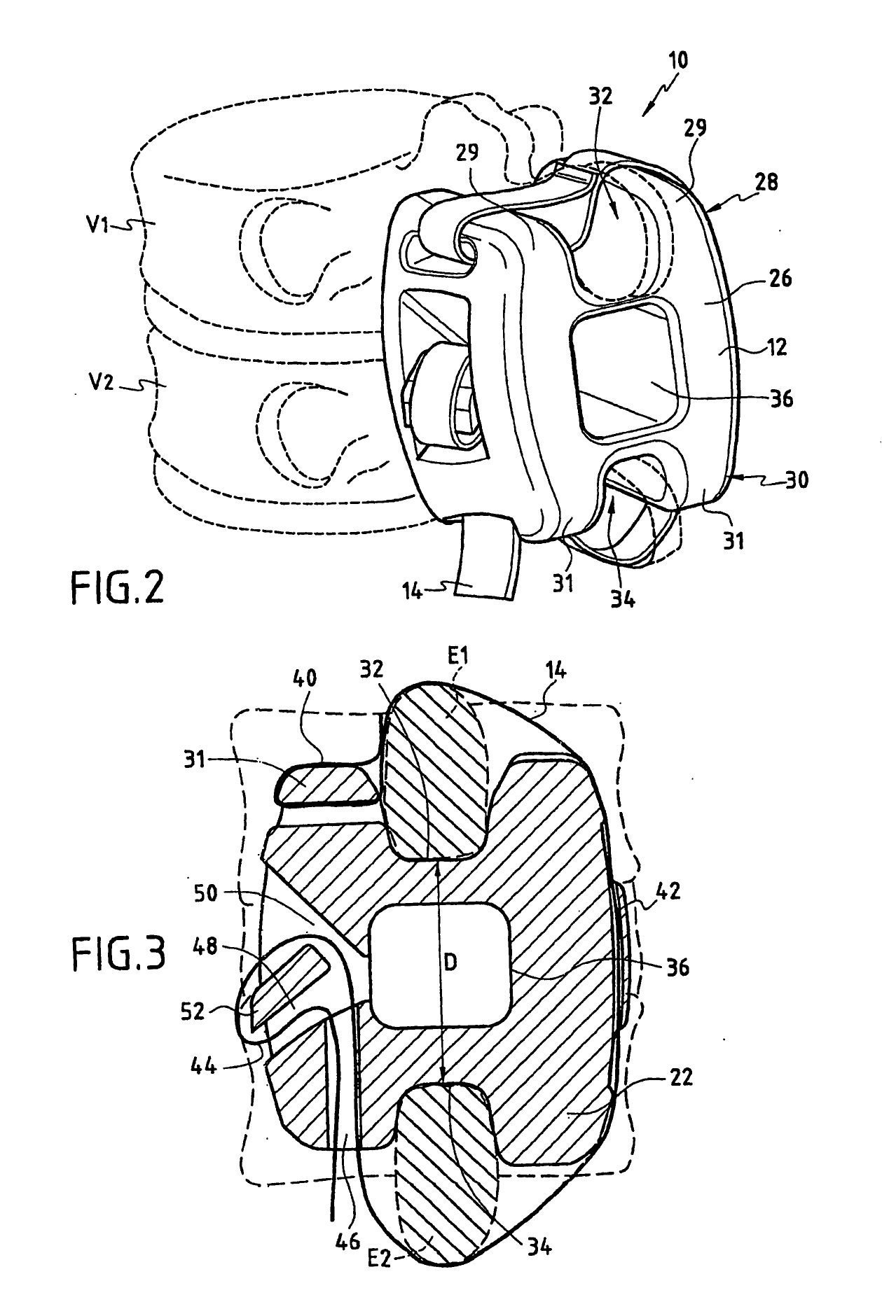

FIGS. 2 and 3 show an intervertebral implant that is particularly well adapted to implementing the invention. However, other types of intervertebral implant could naturally also be used. The implant described corresponds to that of PCT patent application WO 02/051326.

The implant 10 is constituted by a spacer 12 and fastener means 14. The spacer 12 comprises a central portion 26 and two end portions 28 and 30. Each end portion 28 and 30 has a pair of limbs 29 and 31 defining two parallel grooves 32 and 34. Each groove 32, 34 is designed to receive a spinus process E₁, E₂ from the corresponding one of the vertebrae V₁, V₂ between which the spacer 12 is inserted.

The spacer 12, which is made out of a rigid material, preferably includes a recess 36 in its central portion 26, e.g. a recess of rectangular right section, extending parallel to the groves 32 and 34. This recess 36 gives a certain amount of resilience to the central portion 26 of the spacer, thus allowing a certain amount of relative displacement between the vertebrae V₁ and V₂. Nevertheless, on average, the distance between the two vertebrae is determined by the distance D between the bottoms of the grooves 32 and 34.

In order to hold the spinus processes E₁ and E₂ in the grooves 32 and 34 of the spacer 12, the implant includes fastener means constituted in the particular example described by a strip 14.

The strip 14 has a first end 40 which is secured to one limb 29 of the groove 32. The covering portion 42 of the strip 14 passes over the grooves 32 and 34 and its second end 44 passes through slots 46, 48, and 50 formed in the limb 31 and in the middle portion 26 of the spacer. The portion 52 of the spacer defined by the slots 48 and 50 presents a tapering end 54 enabling the strip 14 to be self-locking.

1. An assembly for treating degeneration of a damaged intervertebral disk disposed between two vertebrae, said assembly comprising:
cells optionally analogous to those of the intervertebral disk, and suitable for being implanted in said disk; and
an intervertebral implant comprising:
an intervertebral spacer for placing between said vertebrae in order to limit the stresses applied to said disk; and
fastener means for fastening said spacer to said vertebrae.

2. A treatment assembly according to claim 1, wherein said cells are as obtained by being taken from an intervertebral disk of the patient and culturing said cells as taken.

3. A treatment assembly according to claim 1, wherein said cells are such as obtained by taking autologous adult stem cells from the bone marrow of the patient and by culturing said cells.

4. A treatment assembly according to claim 1, wherein said cells are such as obtained by taking embryonic stem cells and by culturing said cells.

5. A treatment assembly according to claim 1 further comprising injector means for injecting said implantable cells into the intervertebral disk of the patient.

6. A treatment assembly according to claim 1 wherein said spacer of said intervertebral implant comprises a central portion and two end portions, each end portion having a groove defined by two limbs, said grooves being suitable for receiving the spinus process of a vertebra.

7. A treatment assembly according to claim 6, wherein said central portion of the spacer is elastically deformable.

8. A treatment assembly according to claim 6, wherein said fastener means for fastening the intervertebral implant comprise at least one strip securable to said spacer and surrounding the processes of said vertebrae so as to hold said processes in said grooves.

9. A treatment assembly according to claim 7, wherein said fastener means for fastening the intervertebral implant comprise at least one strip securable to said spacer and surrounding the processes of said vertebrae so as to hold said processes in said grooves.

10. A method for healing degeneration of a damaged invertebral disk disposed between two vertebrae, said method including the steps of:
placing an intertebral implant between the processes of said vertebrae for limiting the stresses applied to said disk,
injecting into said disk cells optionally analogous to those of the invertrebal disk and suitable for being implanted in said disk; and
fastening said intravertebral disk implant to said processes of the vertebrae.
